(19) United States
(12) Patent Application Publication
(10) Pub. No.: US 2017/0080084 A1
(43) Pub. Date: Mar. 23, 2017

(54) OIL/SURFACTANT MIXTURES FOR SELF-EMULSIFICATION

(71) Applicant: GlaxoSmithKline Biologicals SA, Rixensart (BE)

(72) Inventors: Luis BRITO, Concord, MA (US); Stephanie Kay DODD, Ayer, MA (US); Derek O'HAGAN, Winchester, MA (US); Manmohan SINGH, Cary, NC (US)

(21) Appl. No.: 15/126,508

(22) PCT Filed: Mar. 17, 2015
(86) PCT No.: PCT/EP2015/055509

§ 371 Date: Sep. 15, 2016

(30) Foreign Application Priority Data
Mar. 17, 2014 (EP) 14160391.0
May 5, 2014 (EP) 14167083.6

(51) Int. Cl. A61K 39/39; C12N 7/00; A61K 47/26; A61K 39/145; A61K 9/107; A61K 47/06

(52) U.S. Cl. CPC A61K 39/39; A61K 9/1075; A61K 47/06; A61K 47/26; A61K 39/145; C12N 7/00; A61K 2039/55566

(57) ABSTRACT

Oil-in-water emulsions with small droplet sizes can be formed without requiring either microfluidisation or heating to cause phase inversion, but rather by simple mixing of a pre-mixed composition of oil and a surfactant component comprising at least one surfactant component with aqueous material. The HLB value of the surfactant component can be selected to give a composition which, on mixing with a volume excess of aqueous material, spontaneously forms an oil in water emulsion with submicron oil droplets having a diameter <250 nm, suitable for filter sterilisation. Droplet diameters of <40 nm can also be achieved.

This application claims the benefit of European Patent Application No. 14160391.0, filed Mar. 17, 2014, and European Patent Application No. 14167083.6, filed May 5, 2014, the complete contents of both of which are hereby incorporated herein by reference for all purposes.

### TECHNICAL FIELD

This invention relates to improved methods of manufacturing oil-in-water emulsions having small oil droplet particle sizes e.g. which are useful as vaccine adjuvants.

### BACKGROUND ART

The vaccine adjuvant known as ‘MF59’ [1-3] is a submicron oil-in-water emulsion of squalene, polysorbate 80 (also known as Tween 80), and sorbitan trioleate (also known as Span 85). It may also include citrate ions e.g. 10 mM sodium citrate buffer. The composition of the emulsion by volume can be about 5% squalene, about 0.5% Tween 80 and about 0.5% Span 85. The adjuvant and its production are described in more detail in references 4 (chapter 10), 5 (chapter 12) and 6 (chapter 19). As described in reference 7, it is manufactured on a commercial scale by dispersing Span 85 in the squalene, dispersing Tween 80 in an aqueous phase (citrate buffer), then mixing these two phases to form a coarse emulsion which is then microfluidised. The emulsion is prepared at double-strength and is diluted 1:1 (by volume) with the relevant vaccine.

The emulsion adjuvant known as ‘AS03’ [8] is prepared by mixing an oil mixture (consisting of squalene and α-tocopherol) with an aqueous phase (Tween 80 and buffer), followed by microfluidisation [9]. It is also prepared at double-strength.

The emulsion adjuvant known as ‘AF03’ is prepared by cooling a pre-heated water-in-oil emulsion until it crosses its emulsion phase inversion temperature, at which point it thermoreversibly converts into an oil-in-water emulsion [10]. The ‘AF03’ emulsion includes squalene, sorbitan oleate, polyoxyethylene cetostearyl ether and mannitol. The mannitol, cetostearyl ether and a phosphate buffer are mixed in one container to form an aqueous phase, while the sorbitan ester and squalene are mixed in another container to form an oily component. The aqueous phase is added to the oily component and the mixture is then heated to ˜60° C. and cooled to provide the final emulsion. The emulsion is initially prepared with a composition of 32.5% squalene, 4.8% sorbitan oleate, 6.2% polyoxyethylene cetostearyl ether and 6% mannitol, which is at least 4× final strength.

As demonstrated above, previous methods known in the art for producing emulsions suitable for use as adjuvants require either vigorous mechanical processes (such as homogenisation and microfluidization) or relatively high temperatures (for example in a phase inversion temperature process) in order achieve the small oil droplet sizes required for adjuvant activity. The use of these processes is associated with several disadvantages e.g. high manufacturing costs.

### SUMMARY OF THE INVENTION

Accordingly, it is an object of the present invention to provide further and improved (e.g. simpler) methods for the production of submicron oil-in-water emulsions. In particular, it is an object of the present invention to provide methods that are suitable for use on a commercial scale and which do not require the use of processes involving vigorous mechanical treatment or significantly elevated temperatures.

The inventors have discovered that oil-in-water emulsions with small droplet sizes can be formed without requiring either microfluidisation or heating to cause phase inversion, but rather by simple mixing of a pre-mixed composition of oil and surfactant with aqueous material. In general, if the oil/surfactant composition contains a greater volume of surfactant than oil then, on mixing with an excess volume of aqueous material the system spontaneously forms an oil-in-water emulsion with submicron oil droplets (and even with droplets having a diameter <250 nm, suitable for filter sterilisation). These emulsions show adjuvant activity even though they have different compositions from known adjuvants.

In a first aspect, the present invention provides a pharmaceutically acceptable oil/surfactant composition for use in the preparation of an oil-in-water emulsion having an average oil particle diameter of less than 250 nm (e.g. between 40-250 nm, between 90-240 nm, e.g. from 100 to 220 nm), said composition comprising
at least one biocompatible, metabolizable oil (such as squalene); and
a biocompatible, metabolizable surfactant component comprising at least one surfactant;
   wherein the surfactant component has an HLB value of from 2.5 to 9.0, and wherein the oil/surfactant composition is substantially free of aqueous components. This oil/surfactant composition contrasts with the squalene/sorbitan trioleate composition which is used for manufacturing MF59 (see FIG. 1 of reference 11) in which the surfactant component has a HLB of less than 2.

In a second aspect the present invention provides an oil-in-water emulsion comprising the oil/surfactant composition according to the first aspect of the invention in combination with an aqueous phase, wherein said emulsion has an average oil particle diameter of less than 250 nm. In general, when compared to microfluidised emulsions having equivalent oil droplet sizes and distributions, such emulsions will have slightly higher surfactant:oil weight ratios e.g more than 1:4.3 for an emulsion similar to MF59, and more than 1:4.7 for an emulsion similar to AS03.

In a third aspect the present invention provides a method of forming an oil-in-water emulsion comprising
at least one biocompatible, metabolizable oil; and
a biocompatible, metabolizable surfactant component comprising at least one surfactant;
   wherein the surfactant component has an HLB value of from 2.5 to 9.0 and wherein the emulsion has an average oil particle diameter of less than 250 nm;
   said method comprising:
providing an oil/surfactant composition according to the first aspect of the invention;
providing an aqueous phase;
combining the oil/surfactant composition with a volume excess of the aqueous phase to form a diluted composition; and
gently mixing the diluted composition to form an oil-in-water emulsion having an average oil particle diameter of less than 250 nm.

In a fourth aspect the present invention provides a pharmaceutically acceptable oil/surfactant composition for use in the preparation of an oil-in-water emulsion having an average oil particle diameter of no greater than 40 nm, said composition comprising
at least one biocompatible, metabolizable oil; and
a biocompatible, metabolizable surfactant component comprising at least one surfactant;
   wherein the surfactant component has an HLB value of from 9.0 to 14.0 and wherein the oil/surfactant composition is substantially free of aqueous components.

In a fifth aspect the present invention provides an oil-in-water emulsion comprising the oil/surfactant composition according to the fourth aspect of the invention in combination with an aqueous phase, wherein said emulsion has an average oil particle diameter of no greater than 40 nm.

In a sixth aspect the present invention provides a method of forming an oil-in-water emulsion comprising
at least one biocompatible, metabolizable oil; and
a biocompatible, metabolizable surfactant component comprising at least one surfactant;
wherein the surfactant component has an HLB value of from 9.0 to 14.0 and wherein the emulsion has an average oil particle diameter of no greater than 40 nm;
   said method comprising:
providing an oil/surfactant composition according to the fourth aspect of the invention;
providing an aqueous phase;
combining the oil/surfactant composition with a volume excess of the aqueous phase to form a diluted composition; and
gently mixing the diluted composition to form an oil-in-water emulsion having an average oil particle diameter of no greater than 40 nm.

The invention also provides oil-in-water emulsions of the invention for use as adjuvants.

In a further aspect the present invention provides an immunogenic composition comprising an oil-in-water emulsion according to the present invention, and an antigen component.

In one aspect the present invention provides a process for preparing an immunogenic composition, said process comprising mixing an oil-in-water emulsion according to the present invention, with an antigen component.

In another aspect the present invention provides a kit comprising:
an oil/surfactant composition according to the present invention;
an aqueous phase; and optionally
instructions for combining the oil/surfactant composition and aqueous phase.

According to embodiments of the invention the oil/surfactant composition and/or the aqueous phase may comprise an antigen component.

In a further aspect the invention provides a process for preparing a kit comprising the steps of: providing an oil/surfactant composition according to the present invention; and packaging the composition into a kit as a kit component together with an aqueous phase; and optionally packaging an antigen component into the kit as a kit component together with the oil/surfactant composition and the aqueous phase.

The present invention also provides a kit comprising: an oil-in-water emulsion according to the present invention; and an antigen component.

The present invention further provides a process for preparing a kit comprising the steps of: providing an oil-in-water emulsion according to the present invention; and packaging the emulsion into a kit as a kit component together with a separate antigen component.

The present invention also provides a dry material (e.g. a lyophilisate) which, when reconstituted with an aqueous phase provides an oil-in-water emulsion according to the present invention. As used herein, “dry material” and “dried material” means material which is substantially free of water or substantially free of an aqueous phase.

The invention also provides a method for preparing a dried emulsion, comprising: (i) obtaining an oil-in-water emulsion of the invention; and (ii) drying the emulsion to provide the dried emulsion. This dried material can be reconstituted into an emulsion of the invention by combining it with a suitable aqueous carrier. Suitable drying techniques are discussed below.

The present invention also provides a kit for preparing an oil-in-water emulsion according to the present invention, wherein the kit comprises:
an oil-in-water emulsion according to the present invention in dried form; and
an aqueous phase.

Embodiments of the present invention are set out in the following numbered clauses:
1. A pharmaceutically acceptable oil/surfactant composition for use in the preparation of an oil-in-water emulsion having an average oil particle diameter of less than 250 nm, said composition comprising
   (i) at least one biocompatible, metabolizable oil; and
   (ii) a biocompatible, metabolizable surfactant component comprising at least one surfactant;
   wherein the surfactant component has an HLB value of from 2.5 to 9.0, and wherein the oil/surfactant composition is substantially free of aqueous components.
2. The oil/surfactant composition according to clause 1, wherein the surfactant component has an HLB value of from 3.3 to 8.4.
3. The oil/surfactant composition according to clause 1 or clause 2, wherein the oil:surfactant component volume ratio is from 1:1 to 1:10.
4. The oil/surfactant composition according to any one of clauses 1 to 3, wherein the surfactant component comprises at least two surfactants.
5. The oil/surfactant composition according to any one of clauses 1 to 4, wherein the surfactant component comprises a first surfactant having an HLB value of less than 10 and a second surfactant having an HLB value of at least 10.
6. The oil/surfactant composition according to any one of clauses 1 to 5, wherein the surfactant component comprises a first surfactant having an HLB value of from 1 to 4 and a second surfactant having an HLB value of from 13 to 17.
7. The oil/surfactant composition according to any one of clauses 1 to 6, wherein the oil/surfactant composition comprises squalene, sorbitan trioleate and polysorbate 80.
8. The oil/surfactant composition according to clause 7, wherein the combined % by vol. of sorbitan trioleate and polysorbate 80 is from 50 to 90% of the total volume of the oil/surfactant composition.
9. The oil/surfactant composition according to clause 7 or clause 8, wherein the combined % by vol. of squalene and polysorbate 80 is from 20 to 70% of the total volume of the oil/surfactant composition.
10. The oil/surfactant composition according to any one of clauses 7 to 9, wherein the combined % by vol. of squalene and sorbitan trioleate is from 60 to 90% of the total volume of the oil/surfactant composition.
11. The oil/surfactant composition according to clause 7, wherein the combined % by vol. of sorbitan trioleate and polysorbate 80 is from 50 to 90% of the total volume of squalene, sorbitan trioleate and polysorbate 80 in the oil/surfactant composition.
12. The oil/surfactant composition according to clause 7 or clause 11, wherein the combined % by vol. of squalene and polysorbate 80 is from 20 to 70% of the total volume of squalene, sorbitan trioleate and polysorbate 80 in the oil/surfactant composition.
13. The oil/surfactant composition according to any one of clauses 7, 11 or 12, wherein the combined % by vol. of squalene and sorbitan trioleate is from 60 to 90% of the total volume of squalene, sorbitan trioleate and polysorbate 80 in the oil/surfactant composition.
14. The oil/surfactant composition according to any one of clauses 1 to 13 selected from:

| Squalene | Sorbitan Trioleate | Polysorbate 80 |
|---|---|---|
| (% by vol.) | (% by vol.) | (% by vol.) |
| 40 | 40 | 20 |
| 30 | 40 | 30 |
| 50 | 30 | 20 |
| 40 | 30 | 30 |

15. An oil-in-water emulsion comprising the oil/surfactant composition of any one of clauses 1 to 14 in combination with an aqueous phase, wherein said emulsion has an average oil particle diameter of less than 250 nm.
16. The oil-in-water emulsion according to clause 15, wherein the emulsion has an average oil particle diameter of from 100 to 220 nm.
17. A method of forming an oil-in-water emulsion comprising
   (i) at least one biocompatible, metabolizable oil; and
   (ii) a biocompatible, metabolizable surfactant component comprising at least one surfactant;
   wherein the surfactant component has an HLB value of from 2.5 to 9.0 and wherein the emulsion has an average oil particle diameter of less than 250 nm;
   said method comprising:
   a) providing an oil/surfactant composition according to any one of clauses 1 to 14;
   b) providing an aqueous phase;
   c) combining the oil/surfactant composition with a volume excess of the aqueous phase to form a diluted composition; and
   d) gently mixing the diluted composition to form an oil-in-water emulsion having an average oil particle diameter of less than 250 nm.
18. The method according to clause 17, wherein the emulsion has an average oil particle diameter of from 100 to 220 nm.
19. The method according to any one of clauses 17 to 18, wherein the method is performed in the absence of mechanical agitation.
20. The method according to any one of clauses 17 to 19, wherein the gentle mixing of the diluted composition comprises inversion of the mixture by hand or by stirring or by a mechanical device.
21. The method according to any one of clauses 17 to 20, wherein the step of combining the oil/surfactant composition with a volume excess of the aqueous phase to form a diluted composition comprises:
   (i) mixing equal volumes of oil/surfactant composition and aqueous phase; and
   (ii) diluting the mixture of oil/surfactant composition and aqueous phase with a further volume of aqueous phase to form a diluted composition.
22. The method according to any one of clauses 17 to 21 wherein the step of combining the oil/surfactant composition with a volume excess of the aqueous phase to form a diluted composition comprises adding the oil/surfactant composition into the aqueous phase.
23. The method according to any one of clauses 17 to 22 wherein the oil/surfactant composition is added to a volume excess of the aqueous phase at a temperature below 55° C.
24. The method according to any one of clauses 17 to 23 wherein the process takes place at a temperature of about 25° C.
25. The method according to any one of clauses 17 to 24, wherein the ratio of aqueous phase to oil/surfactant composition in the diluted composition is from 10:1 to 50:1 by volume, preferably 20:1 to 40:1 by volume.
26. The method according to any one of clauses 17 to 25, wherein said method further comprises the step of subjecting the oil-in-water emulsion to filter sterilisation.
27. The method according to any one of clauses 17 to 26, wherein the aqueous phase is substantially free from surfactant(s) and/or oil(s).
28. The oil-in-water emulsion according to clause 15 or clause 16, for use as an adjuvant.
29. An immunogenic composition comprising an oil-in-water emulsion according to clause 15 or clause 16, and an antigen component.
30. A process for preparing an immunogenic composition comprising mixing an oil-in-water emulsion according to clause 15 or clause 16, with an antigen component.
31. A kit comprising:
   (i) an oil/surfactant composition according to any one of clauses 1 to 14;
   (ii) an aqueous phase; and optionally
   (iii) instructions for combining the oil/surfactant composition and aqueous phase.
32. The kit according to clause 31, wherein the oil/surfactant composition further comprises an antigen component.
33. The kit according to clause 31 or clause 32, wherein the aqueous phase further comprises an antigen component.
34. A process for preparing a kit comprising the steps of:
   (i) providing an oil/surfactant composition according to any one of clauses 1 to 14; and
   (ii) packaging the composition into a kit as a kit component together with an aqueous phase.
35. A process for preparing a kit according to clause 34, further comprising:
   (iii) packaging an antigen component into the kit as a kit component together with the oil/surfactant composition and the aqueous phase.
36. A kit comprising:
   (i) an oil-in-water emulsion according to clause 15 or clause 16; and
   (ii) an antigen component.
37. A process for preparing a kit comprising the steps of:
   (i) providing an oil-in-water emulsion according to clause 15 or clause 16; and
   (ii) packaging the emulsion into a kit as a kit component together with a separate antigen component.
38. A dried material which, when reconstituted with an aqueous phase provides an oil-in-water emulsion according to clause 15 or clause 16.
39. The dried material according to clause 38, further comprising an antigen component in dried form.
40. The dried material according to clause 38 or clause 39, further comprising one or more lyophilisation stabilisers.
41. A kit for preparing an oil-in-water emulsion according to clause 15 or clause 16, wherein the kit comprises:
   (i) an oil-in-water emulsion according to clause 15 or clause 16 in dried form; and
   (ii) an aqueous phase.
42. The kit according to clause 41, wherein the oil-in-water emulsion in dried form is combined with an antigen component in dried form.
43. A pharmaceutically acceptable oil/surfactant composition for use in the preparation of an oil-in-water emulsion having an average oil particle diameter of no greater than 40 nm, said composition comprising
   (i) at least one biocompatible, metabolizable oil; and
   (ii) a biocompatible, metabolizable surfactant component comprising at least one surfactant;
   wherein the surfactant component has an HLB value of from 9.0 to 14.0 and wherein the oil/surfactant composition is substantially free of aqueous components.
44. The oil/surfactant composition according to clause 43, wherein the surfactant component has an HLB value of from 9.3 to 13.5.
45. The oil/surfactant composition according to clause 43 or clause 44, wherein the oil:surfactant component volume ratio is from 1:1 to 1:10.
46. The oil/surfactant composition according to any one of clauses 43 to 45, wherein the surfactant component comprises at least two surfactants.
47. The oil/surfactant composition according to any one of clauses 43 to 46, wherein the surfactant component comprises a first surfactant having an HLB value of less than 10 and a second surfactant having an HLB value of at least 10.
48. The oil/surfactant composition according to any one of clauses 43 to 47, wherein the surfactant component comprises a first surfactant having an HLB value of from 1 to 4 and a second surfactant having an HLB value of from 13 to 17
49. The oil/surfactant composition according to any one of clauses 43 to 48, wherein the oil/surfactant composition comprises squalene, sorbitan trioleate and polysorbate 80.
50. The oil/surfactant composition according to clause 49, wherein the combined % by vol. of sorbitan trioleate and polysorbate 80 is from 60 to 90% of the total volume of the oil/surfactant composition.
51. The oil/surfactant composition according to clause 49 or clause 50, wherein the combined % by vol. of squalene and polysorbate 80 is from 70 to 90% of the total volume of the oil/surfactant composition.
52. The oil/surfactant composition according to any one of clauses 49 to 51, wherein the combined % by vol. of squalene and sorbitan trioleate is from 20 to 60% of the total volume of the oil/surfactant composition.
53. The oil/surfactant composition according to clause 49, wherein the combined % by vol. of sorbitan trioleate and polysorbate 80 is from 60 to 90% of the total volume of squalene, sorbitan trioleate and polysorbate 80 in the oil/surfactant composition.
54. The oil/surfactant composition according to clause 49 or clause 53, wherein the combined % by vol. of squalene and polysorbate 80 is from 70 to 90% of the total volume of squalene, sorbitan trioleate and polysorbate 80 in the oil/surfactant composition.
55. The oil/surfactant composition according to any one of clauses 49, 53 or 54, wherein the combined % by vol. of squalene and sorbitan trioleate is from 20 to 60% of the total volume of squalene, sorbitan trioleate and polysorbate 80 in the oil/surfactant composition.
56. The oil/surfactant composition according to any one of clauses 43 to 55 selected from:

| Squalene | Sorbitan Trioleate | Polysorbate 80 |
|---|---|---|
| (% by vol.) | (% by vol.) | (% by vol.) |
| 30 | 30 | 40 |
| 40 | 20 | 40 |
| 30 | 20 | 50 |
| 10 | 10 | 80 |

57. An oil-in-water emulsion comprising the oil/surfactant composition of any one of clauses 43 to 56 in combination with an aqueous phase, wherein said emulsion has an average oil particle diameter of no greater than 40 nm.
58. The oil-in-water emulsion according to clause 57, wherein the emulsion has an average oil particle diameter of no greater than 30 nm.
59. A method of forming an oil-in-water emulsion comprising
   (i) at least one biocompatible, metabolizable oil; and
   (ii) a biocompatible, metabolizable surfactant component comprising at least one surfactant;
   wherein the surfactant component has an HLB value of from 9.0 to 14.0 and wherein the emulsion has an average oil particle diameter of no greater than 40 nm;
   said method comprising:
   a) providing an oil/surfactant composition according to any one of clauses 43 to 56;
   b) providing an aqueous phase;
   c) combining the oil/surfactant composition with a volume excess of the aqueous phase to form a diluted composition; and
   d) gently mixing the diluted composition to form an oil-in-water emulsion having an average oil particle diameter of no greater than 40 nm.
60. The method according to clause 59, wherein the emulsion has an average oil particle diameter of no greater than 30 nm.
61. The method according to clause 59 or clause 60, wherein the method is performed in the absence of mechanical agitation.
62. The method according to any one of clauses 59 to 61, wherein the gentle mixing of the diluted composition comprises inversion of the mixture by hand or by stirring or by a mechanical device.
63. The method according to any one of clauses 59 to 62, wherein the step of combining the oil/surfactant composition with a volume excess of the aqueous phase to form a diluted composition comprises:
   (i) mixing equal volumes of oil/surfactant composition and aqueous phase; and
   (ii) diluting the mixture of oil/surfactant composition and aqueous phase with a further volume of aqueous phase to form a diluted composition.
64. The method according to any one of clauses to 59 to 63 wherein the step of combining the oil/surfactant composition with a volume excess of the aqueous phase to form a diluted composition comprises adding the oil/surfactant composition into the aqueous phase.
65. The method according to any one of clauses 59 to 64, wherein the oil/surfactant composition is added to a volume excess of the aqueous phase at a temperature below 55° C.
66. The method according to any one of clauses 59 to 65, wherein the process takes place at a temperature of about 25° C.
67. The method according to any one of clauses 59 to 66, wherein the ratio of aqueous phase to oil/surfactant composition in the diluted composition is from 10:1 to 50:1 by volume, preferably from 20:1 to 40:1 by volume.
68. The method according to any one of clauses 59 to 67, wherein said method further comprises the step of subjecting the oil-in-water emulsion to filter sterilisation.
69. The method according to any one of clauses 59 to 68, wherein the aqueous phase is substantially free from surfactant(s) and/or oil(s).

### The Oil/Surfactant Composition

According to the invention, processes for preparing emulsions make use of an oil/surfactant composition. This composition is a mixture which includes at least one oil and a surfactant component comprising at least one surfactant, examples of which are discussed in more detail below. The oil(s) and surfactant(s) are ideally miscible in each other in the composition. The composition may be an oil/surfactant dispersion. If the oil and surfactant phases are fully miscible in each other the composition will be an oil/surfactant solution.

Because emulsions of the invention are intended for pharmaceutical use, the oil(s) and the surfactant(s) in the composition will typically be metabolizable (biodegradable) and biocompatible. If only one of these two components is metabolizable and biocompatible, it should be the oil(s).

The composition can include component(s) in addition to the oil(s) and surfactant(s), but in some embodiments the oil(s) and surfactant(s) make up substantially all of the composition. When further components are included, they may form up to 15% of the composition (by weight), preferably 1-15% of the composition (by weight), more preferably 5-10%. For instance, in some embodiments the composition includes one or more pharmacologically active agent(s), which will usually be lipophilic. Such lipophilic agents include, but are not limited to, vitamins (e.g. vitamins A, D, E and K), carotenoids (e.g. β-carotene), fatty acids (e.g. arachidonic acid, ecosapentaenoic acid docosahexaenoic acid), pyrimidines (e.g. 5-hydroxy-4,6-dimethyl-2-(6-phenylhexyl) aminopyrimidine), ansamitocins, angiotensin II receptor antagonists (e.g. candesartan cilexetil), and immunopotentiators (e.g. muramyl dipeptides). Typical lipophilic agents have a positive log P value (partition coefficient measured in 1-octanol and water) at pH 7.4 and 37° C. e.g. they may have a log P value ≧1, ≧2, ≧3, ≧4, ≧5, ≧6, ≧7, etc.

In some embodiments the composition can include a cholesterol and/or a phospholipid. Suitable classes of phospholipid include, but are not limited to, phosphatidylethanolamines, phosphatidylcholines, phosphatidylserines, phosphatidylglycerols, etc.

In embodiments of the invention, the oil/surfactant composition may comprise an antigen component.

In a first aspect, the present invention provides a pharmaceutically acceptable oil/surfactant composition for use in the preparation of an oil-in-water emulsion having an average oil particle diameter of less than 250 nm, said composition comprising
at least one biocompatible, metabolizable oil; and
a biocompatible, metabolizable surfactant component comprising at least one surfactant;
   wherein the surfactant component has an HLB value of from 2.5 to 9.0, and wherein the oil/surfactant composition is substantially free of aqueous components.

In a fourth aspect the present invention provides a pharmaceutically acceptable oil/surfactant composition for use in the preparation of an oil-in-water emulsion having an average oil particle diameter of no greater than 40 nm, said composition comprising
at least one biocompatible, metabolizable oil; and
a biocompatible, metabolizable surfactant component comprising at least one surfactant;
   wherein the surfactant component has an HLB value of from 9.0 to 14.0 and wherein the oil/surfactant composition is substantially free of aqueous components.

The oil:surfactant component volume ratio in the oil/surfactant composition of the present invention is preferably from 1:1 to 1:10.

Preferably, the oil/surfactant composition of the present invention comprises an oil, a surfactant having an HLB value of from 13 to 17, and a surfactant having an HLB value of from 1 to 5. More preferably the oil/surfactant composition of the present invention consists essentially of an oil, a surfactant having an HLB value of from 13 to 17, and a surfactant having an HLB value of from 1 to 5. In some embodiments the oil/surfactant composition consists of an oil, a surfactant having an HLB value of from 13 to 17, and a surfactant having an HLB value of from 1 to 5.

According to embodiments of the first aspect of the present invention wherein the oil/surfactant composition comprises an oil, a surfactant having an HLB value of from 13 to 17, and a surfactant having an HLB value of from 1 to 5:
the combined % by volume of surfactant having an HLB value of from 1 to 5 and surfactant
having an HLB value of from 13 to 17 in the oil/surfactant composition may be from 50 to 90% preferably 50-70% of the total volume of the oil/surfactant composition; and/or
the combined % by volume of oil and surfactant having an HLB value of from 13 to 17 may be from 20 to 70% preferably 60-70% of the total volume of the oil/surfactant composition; and/or
the combined % by volume of oil and surfactant having an HLB value of from 1 to 5 may be from 60 to 90% preferably 70-80% of the total volume of the oil/surfactant composition.

Preferably the oil/surfactant compositions according to embodiments of the first aspect of the present invention satisfy at least two of these three criteria. More preferably the oil/surfactant compositions according to embodiments of the first aspect of the invention satisfy all three of these criteria.

According to embodiments of the first aspect of the present invention wherein the oil/surfactant composition comprises an oil, a surfactant having an HLB value of from 13 to 17, and a surfactant having an HLB value of from 1 to 5:
the combined % by volume of surfactant having an HLB value of from 1 to 5 and surfactant having an HLB value of from 13 to 17 may be from 50 to 90% preferably 50-70% of the total volume of oil, surfactant having an HLB value of from 1 to 5 and surfactant having an HLB value of from 13 to 17 in the oil/surfactant composition; and/or
the combined % by volume of oil and surfactant having an HLB value of from 13 to 17 may be from 20 to 70% preferably 60-70% of the total volume of oil, surfactant having an HLB value of from 1 to 5 and surfactant having an HLB value of from 13 to 17 in the oil/surfactant composition; and/or
the combined % by volume of oil and surfactant having an HLB value of from 1 to 5 may be from 60 to 90% preferably 70-80% of the total volume of oil, surfactant having an HLB value of from 1 to 5 and surfactant having an HLB value of from 13 to 17 in the oil/surfactant composition.

Preferably the oil/surfactant compositions according to embodiments of the first aspect of the present invention satisfy at least two of these three criteria. More preferably the oil/surfactant compositions according to embodiments the first aspect of the invention satisfy all three of these criteria.

In a particular embodiment, the oil/surfactant composition of the present invention comprises squalene, sorbitan trioleate and polysorbate 80. In another embodiment the oil/surfactant composition of the present invention comprises essentially of squalene, sorbitan trioleate and polysorbate 80. In embodiments of the present invention the oil/surfactant composition consists of squalene, sorbitan trioleate and polysorbate 80.

According to embodiments of the first aspect of the present invention wherein the oil/surfactant composition comprises squalene, sorbitan trioleate and polysorbate 80:
the combined % by volume of sorbitan trioleate and polysorbate 80 in the oil/surfactant composition may be from 50 to 90% preferably 50-70% of the total volume of the oil/surfactant composition; and/or
the combined % by volume of squalene and polysorbate 80 may be from 20 to 70% preferably 60-70% of the total volume of the oil/surfactant composition; and/or
the combined % by volume of squalene and sorbitan trioleate may be from 60 to 90% preferably 70-80% of the total volume of the oil/surfactant composition.

Preferably the oil/surfactant compositions according to embodiments of the first aspect of the present invention satisfy at least two of these three criteria. More preferably the oil/surfactant compositions according to embodiments of the first aspect of the invention satisfy all three of these criteria.

According to embodiments of the first aspect of the present invention wherein the oil/surfactant composition comprises squalene, sorbitan trioleate and polysorbate 80:
the combined % by volume of sorbitan trioleate and polysorbate 80 may be from 50 to 90% preferably 50-70% of the total volume of squalene, sorbitan trioleate and polysorbate 80 in the oil/surfactant composition; and/or
the combined % by volume of squalene and polysorbate 80 may be from 20 to 70% preferably 60-70% of the total volume of squalene, sorbitan trioleate and polysorbate 80 in the oil/surfactant composition; and/or
the combined % by volume of squalene and sorbitan trioleate may be from 60 to 90% preferably 70-80% of the total volume of squalene, sorbitan trioleate and polysorbate 80 in the oil/surfactant composition.

Preferably the oil/surfactant compositions according to embodiments of the first aspect of the present invention satisfy at least two of these three criteria. More preferably the oil/surfactant compositions according to embodiments the first aspect of the invention satisfy all three of these criteria.

According to certain embodiments of the first aspect of the present invention, the oil/surfactant composition is selected from:

| | Squalene | Sorbitan trioleate | Polysorbate 80 |
|---|---|---|---|
| | (% by vol.) | (% by vol.) | (% by vol.) |
| 5 | 40 | 40 | 20 |
| 6 | 30 | 40 | 30 |
| 10 | 50 | 30 | 20 |
| 11 | 40 | 30 | 30 |

According to embodiments of the fourth aspect of the present invention wherein the oil/surfactant composition comprises an oil, a surfactant having an HLB value of from 13 to 17, and a surfactant having an HLB value of from 1 to 5:
the combined % by volume of surfactant having an HLB value of from 1 to 5 and surfactant having an HLB value of from 13 to 17 in the oil/surfactant composition may be from 60 to 90% of the total volume of the oil/surfactant composition; and/or
the combined % by volume of oil and surfactant having an HLB value of from 13 to 17 in the oil/surfactant composition may be from 70 to 90% of the total volume of the oil/surfactant composition; and/or
the combined % by volume of oil and surfactant having an HLB value of from 1 to 5 in the oil/surfactant composition may be from 20 to 60% of the total volume of the oil/surfactant composition.

Preferably the oil/surfactant compositions according to embodiments of the fourth aspect of the present invention satisfy at least two of these three criteria. More preferably the oil/surfactant compositions according to embodiments of the fourth aspect of the invention satisfy all three of these criteria.

According to embodiments of the fourth aspect of the present invention wherein the oil/surfactant composition comprises an oil, a surfactant having an HLB value of from 13 to 17, and a surfactant having an HLB value of from 1 to 5:
the combined % by volume of surfactant having an HLB value of from 1 to 5 and surfactant having an HLB value of from 13 to 17 may be from 60 to 90% of the total volume of oil, surfactant having an HLB value of from 1 to 5 and surfactant having an HLB value of from 13 to 17 in the oil/surfactant composition; and/or
the combined % by volume of oil and surfactant having an HLB value of from 13 to 17 may be from 70 to 90% of the total volume of oil, surfactant having an HLB value of from 1 to 5 and surfactant having an HLB value of from 13 to 17 in the oil/surfactant composition; and/or
the combined % by volume of oil and surfactant having an HLB value of from 1 to 5 may be from 20 to 60% of the total volume of oil, surfactant having an HLB value of from 1 to 5 and surfactant having an HLB value of from 13 to 17 in the oil/surfactant composition.

Preferably the oil/surfactant compositions according to embodiments of the fourth aspect of the present invention satisfy at least two of these three criteria. More preferably the oil/surfactant compositions according to embodiments the fourth aspect of the invention satisfy all three of these criteria.

According to embodiments of the fourth aspect of the present invention wherein the oil/surfactant composition comprises squalene, sorbitan trioleate and polysorbate 80:
the combined % by volume of sorbitan trioleate and polysorbate 80 may be from 60 to 90% of the total volume of the oil/surfactant composition; and/or
the combined % by volume of squalene and polysorbate 80 is from 70 to 90% of the total volume of the oil/surfactant composition; and/or
the combined % by volume of squalene and sorbitan trioleate is from 20 to 60% of the total volume of the oil/surfactant composition.

Preferably the oil/surfactant compositions according to embodiments of the fourth aspect of the present invention satisfy at least two of these three criteria. More preferably the oil/surfactant compositions according to embodiments of the fourth aspect of the invention satisfy all three of these criteria.

According to embodiments of the fourth aspect of the present invention wherein the oil/surfactant compositions comprises squalene, sorbitan trioleate and polysorbate 80:
the combined % by volume of sorbitan trioleate and polysorbate 80 is from 60 to 90% of the total volume of squalene, sorbitan trioleate and polysorbate 80 in the oil/surfactant composition; and/or
the combined % by volume of squalene and polysorbate 80 is from 70 to 90% of the total volume of squalene, sorbitan trioleate and polysorbate 80 in the oil/surfactant composition; and/or
the combined % by volume of squalene and sorbitan trioleate is from 20 to 60% of the total volume of squalene, sorbitan trioleate and polysorbate 80 in the oil/surfactant composition.

Preferably the oil/surfactant compositions according to embodiments of the fourth aspect of the present invention satisfy at least two of these three criteria. More preferably the oil/surfactant compositions according to embodiments of the fourth aspect of the invention satisfy all three of these criteria.

According to certain embodiments of the fourth aspect of the present invention, the oil/surfactant composition is selected from:

| | Squalene | Sorbitan Trioleate | Polysorbate 80 |
|---|---|---|---|
| | (% by vol.) | (% by vol.) | (% by vol.) |
| 12 | 30 | 30 | 40 |
| 19 | 40 | 20 | 40 |
| 20 | 30 | 20 | 50 |
| 29 | 10 | 10 | 80 |

In embodiments of the invention, the oil/surfactant composition is substantially free from aqueous components.

### The Oil Component(s)

The composition includes one or more oils. Suitable oil(s) include those from, for example, an animal (such as fish) or a vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and so may be used. The procedures for separation, purification, saponification and other means necessary for obtaining pure oils from animal sources are well known in the art.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. A preferred oil for use with the invention is squalene, which is a branched, unsaturated terpenoid ([(CH₃)₂C[═CHCH₂CH₂C(CH₃)]₂═CHCH₂—]₂; C₃₀H₅₀; 2,6,10,15,19,23-hexamethyl-2,6,10,14,18,22-tetracosahexaene; CAS RN 7683-64-9). Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

The composition of the present invention may comprise a combination of oils e.g. squalene and at least one further oil. Where the composition includes more than one oil, these can be present at various ratios e.g. between 1:5 and 5:1 by volume e.g. between 1:2 and 2:1, such as at equal volumes.

Within the composition, the total amount of oil ideally makes up no more than 50% (by volume). As shown in the examples, higher oil content tends not to favour spontaneous emulsification. In a particular embodiment, preferred amounts of oil in the composition are between 10-50%, more preferably between 25-50%. An oil content in the composition of about 20%, or 30% or 40% (by volume) is particularly useful.

Within the composition, the ratio of the total amount of oil to the total amount of surfactant is ideally no more than 1:1. In a particular embodiment, surfactant is present at a volume excess relative to oil. As shown in the examples, excess oil content tends not to favour spontaneous emulsification. In a particular embodiment, an oil:surfactant volume ratio from 1:1 to 1:10. The oil(s) in the compositions and emulsions of the invention will typically be metabolizable (biodegradable) and biocompatible.

### The Surfactant Component(s)

The composition includes a surfactant component comprising one or more surfactants. In a particular embodiment, the surfactant component comprises at least two surfactants. In a further embodiment, the surfactant component consists of two surfactants. The surfactant component in the compositions and emulsions of the invention will typically be metabolizable (biodegradable) and biocompatible.

Surfactants can be classified by their ‘HLB’ (Griffin's hydrophile/lipophile balance), where a HLB in the range 1 to 10 generally means that the surfactant is more soluble in oil than in water, and a HLB in the range 10 to 20 means that the surfactant is more soluble in water than in oil. HLB values are readily available for surfactants of interest e.g. polysorbate 80 (‘Tween 80’) has a HLB of 15.0 and sorbitan trioleate (‘Span 85’) has a HLB of 1.8.

When two or more surfactants are blended, the resulting HLB of the blend is easily calculated by the weighted average e.g. a 70/30 wt % mixture of polysorbate 80 and sorbitan trioleate has a HLB of (15.0×0.70)+(1.8×0.30) i.e. 11.04.

According to the first, second and third aspects of the present invention the surfactant component comprises at least one surfactant and the surfactant component has an HLB value of from 2.5 to 9.0, preferably from 3.3 to 8.4. For example, the surfactant component may have an HLB value of from 3.5 to 7.7, from 4.0 to 7.5, from 4.7 to 7.1 or from 5.1 to 6.2.

According to the fourth, fifth and sixth aspects of the present invention the surfactant component comprises at least one surfactant and the surfactant component has an HLB value of from 9.0 to 14.0, preferably from 9.3 to 13.5. For example, the surfactant component may have an HLB value of from 10.1 to 11.2. In some embodiments, it can even be possible to prepare an emulsion of the invention using a surfactant component with a HLB above 14.0 e.g. using polysorbate(s) alone. Thus, for instance, an emulsion similar to AS03 could be prepared by applying the methods of the invention to this emulsion's known non-aqueous components (namely squalene, α-tocopherol, and polysorbate 80), but with a slightly higher surfactant:oil weight ratio than in AS03. This could be achieved by increasing the proportion of surfactant (polysorbate 80) relative to oil and/or by decreasing the proportion of oil (squalene and/or α-tocopherol) relative to surfactant.

Where the surfactant component includes more than one surfactant then at least one of them will typically have a HLB of at least 10 (e.g. in the range 12 to 16, or 13 to 17) and at least one has a HLB below 10 (e.g. in the range of 1 to 9, or 1 to 4). For instance, the surfactant component of the composition can include polysorbate 80 and sorbitan trioleate. In embodiments of the present invention the surfactant component comprises a first surfactant having an HLB value of from 1 to 5, preferably 1 to 4 and a second surfactant having an HLB value of from 13 to 17.

As shown in the examples of the application, oil/surfactant compositions comprising a surfactant component having a HLB value in the range of from 2.5 to 9.0 tend to form oil-in-water emulsions having an average oil particle diameter of less than 250 nm e.g. in the range of 100 to 220 nm. Oil/surfactant compositions comprising a surfactant component having a HLB value in the range of from 9.0 to 14.0 tend to form emulsions having an even smaller average oil particle diameter of no greater than 40 nm, more preferably no greater than 30 nm.

The invention can be used with various surfactants, including ionic, non-ionic and zwitterionic surfactants. Non-ionic surfactants are preferred. The invention can thus use surfactants including, but not limited to: the polyoxyethylene sorbitan esters surfactants (commonly referred to as the Tweens or polysorbates), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Many examples of pharmaceutically acceptable surfactants are known in the art for use in the composition and thus in the final emulsion.

In a particular embodiment surfactants for including in the composition are polysorbate 80 (polyoxyethylene sorbitan monooleate) and sorbitan trioleate. If the composition includes a single surfactant then this is a surfactant having an HLB value of 13 to 17, preferably polysorbate 80 (HLB value of 15). If the composition includes two surfactants, a mixture of polysorbate 80 and sorbitan trioleate is preferred. In general terms, compositions including from 20-80% (vol.) sorbitan trioleate have good emulsion forming ability, whereas compositions with only 10% (vol.) sorbitan trioleate performed less well. Similarly, compositions including from 20-80% (vol.) polysorbate 80 have good emulsion forming ability, whereas compositions with only 10% (vol.) polysorbate 80 generally performed less well, although their behaviour could be improved by ensuring at least a 4-fold volume excess of sorbitan trioleate.

Another useful surfactant mixture (as seen in ‘AF03’) is made of sorbitan oleate and polyoxyethylene cetostearyl ether. Sorbitan monooleate has a HLB of 4.3 whereas polyoxyethylene cetostearyl ether has a HLB 13.5.

Within the composition, the total amount of surfactant ideally makes up at least 50% (by volume). As shown in the examples, lower surfactant content tends not to favour spontaneous emulsification. In a particular embodiment, the amounts of surfactant in the composition are between 50-90%. In a further embodiment, the amounts of surfactant in the composition are between 60-90%. A surfactant content in the composition of about 60%, 70%, 80% or 90% (by volume) is particularly useful.

Where an oil/surfactant composition includes a surfactant having a HLB above 8 then the concentration of that surfactant is preferably at least 4000× higher than its critical micelle concentration (CMC) e.g. at least 5000× higher, 6000× higher, 8000× higher, 10000× higher, 12000× higher, 15000× higher, or even at least 20000× higher than the CMC.

The ratio of the total amount of surfactant to the total amount of oil is ideally at least 1:1. In a particular embodiment, surfactant is present at a volume excess relative to oil. As shown in the examples, excess oil content tends not to favour spontaneous emulsification. In a particular embodiment, a surfactant:oil volume ratio is from 1:1 to 10:1.

### Squalene-Containing Oil/Surfactant Mixtures

In a particularly preferred embodiment, the oil for use with the invention is squalene (which will usually be used as the sole oil component, but can optionally be used in combination with another oil such as an α-tocopherol). In combination with a surfactant component comprising or consisting of polysorbate 80 (such as a mixture of polysorbate 80 and sorbitan trioleate) squalene gives useful compositions.

The surfactant component in these compositions can have an HLB value of from 10 to 18 (for example from 13 to 17, or in particular embodiments from 14 to 16), or a high-HLB surfactant can be mixed with a lower HLB surfactant to provide a surfactant component with an HLB value of from 6 to 11 or from 2.5 to 9.0 (e.g. from 3.3 to 8.4). The surfactant component can comprise a first surfactant having an HLB value of less than 10 and a second surfactant having an HLB value of at least 10. For instance it can comprise a first surfactant having an HLB value of from 1 to 4 (e.g. sorbitan trioleate) and a second surfactant having an HLB value of from 13 to 17 (e.g. polysorbate 80).

Ideally these oil/surfactant compositions have a volume excess of squalene relative to the surfactant component. Thus, for instance, it can include (by volume): 60-80% squalene, with the remainder being either polysorbate 80 or a mixture of polysorbate 80 and sorbitan trioleate. For example, the amount of squalene can be 65-75%, 68-72% squalene, 69-71% squalene, or 70% squalene by volume.

Ideally these compositions consist essentially of only these two or three components i.e. squalene, polysorbate 80 and, optionally, sorbitan trioleate. For instance, ≧95% by volume of the composition is made up of (a) squalene and polysorbate 80 or (b) squalene, polysorbate 80, and sorbitan trioleate. A composition consisting of squalene and polysorbate 80 can be used, ideally with a volume excess of squalene.

The combined % by volume of squalene and polysorbate 80 in these compositions can be from 70 to 90% of the total volume of the oil/surfactant composition. Where the composition includes squalene, sorbitan trioleate and polysorbate 80, the combined % by volume of squalene and polysorbate 80 can be from 70 to 90% of the total volume of squalene, sorbitan trioleate and polysorbate 80 in the oil/surfactant composition.

In more general terms, the combined % by volume of oil and a surfactant having an HLB value of from 13 to 17 may be from 70 to 90% of the total volume of oil, the surfactant having an HLB value of from 1 to 5 and the surfactant having an HLB value of from 13 to 17 in the oil/surfactant composition.

Instead of polysorbate 80 the composition can use another non-ionic surfactant having a HLB in the range of 14-16, but polysorbate 80 is preferred.

### The Aqueous Phase

According to the invention, processes for preparing emulsions make use of an aqueous phase. This aqueous phase can be plain water (e.g. w.f.i.) or can include further components e.g. solutes. For instance, in particular embodiments, it preferably includes salts, which can be used to influence tonicity and/or to control pH. For instance, the salts can form a pH buffer e.g. citrate or phosphate salts, such as sodium salts. Typical buffers include: a phosphate buffer; a Tris buffer; a borate buffer; a succinate buffer; a histidine buffer; or a citrate buffer. In preferred embodiments the aqueous phase is free from buffers. In other embodiments of the invention a buffered aqueous phase is preferred, and buffers will typically be included in the 1-20 mM range.

The aqueous phase may suitably be buffered. Any physiologically acceptable buffer may be used herein, such as water, citrate buffers, phosphate buffers, acetate buffers, tris buffers, bicarbonate buffers, carbonate buffers, succinate buffer, or the like. The pH of the aqueous phase will preferably be between 6.0-8.0, preferably about 6.2 to about 6.8. In an exemplary embodiment, the buffer is 10 mM citrate buffer with a pH at 6.5. The aqueous phase may comprise pickering agents such as mannitol to reduce superficial tension.

The aqueous phase can include solutes for influencing tonicity and/or osmolality. The tonicity can be selected to be isotonic with human tissues. To control tonicity, the emulsion may comprise a physiological salt, such as a sodium salt. Sodium chloride (NaCl), for example, may be used at about 0.9% (w/v) (physiological saline). Other salts that may be present include potassium chloride, potassium dihydrogen phosphate, disodium phosphate, magnesium chloride, calcium chloride, etc. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the present invention. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents useful in the present invention. Non-ionic tonicity modifying agents can be present at a concentration of from about 0.1% to about 10% or about 1% to about 10%, of the aqueous phase depending upon the agent that is used.

The aqueous phase ideally has a pH between 5.5 and 8.5 e.g. between 6.0 and 8.0, or between 6.5 and 7.5. This pH range maintains compatibility with normal physiological conditions and, in certain instances, may be required in order to ensure stability of certain components of the emulsion.

In particular embodiments, the aqueous phase is substantially free from oil(s). Thus, on mixing with the oil/surfactant composition to form an emulsion, substantially all of the oil in the emulsion should be sourced from the composition. In another embodiment, the aqueous phase is substantially free from surfactant(s). Thus, on mixing with the oil/surfactant composition to form an emulsion, substantially all of the surfactant in the emulsion should be sourced from the composition. In a further embodiment, the aqueous phase is substantially free from both oil(s) and surfactant(s).

In embodiments of the invention, the aqueous phase may comprise an antigen component.

### Mixing

Unlike MF59 and AS03, emulsions of the invention can be prepared without requiring the use of homogenisers or microfluidisers. Unlike AF03, emulsions of the invention can be prepared without requiring heating up to >50° C. Instead, mixing the oil/surfactant composition with the aqueous phase can lead to spontaneous formation of a submicron emulsion even with only gentle agitation/mixing (e.g. simple manual inversion).

The present invention provides a method of forming an oil-in-water emulsion according to the invention, said method comprising:
providing an oil/surfactant composition according to the invention;
providing an aqueous phase;
combining the oil/surfactant composition with a volume excess of the aqueous phase to form a diluted composition; and
gently mixing the diluted composition to form an oil-in-water emulsion.

The step of forming a diluted composition can take place by simple mixing of the oil/surfactant composition with the aqueous phase. Preferably the formation of a diluted composition is achieved by adding the oil/surfactant composition into the aqueous phase. The step of combining the oil/surfactant composition with a volume excess of the aqueous phase to form a diluted composition may sometimes comprise two separate steps: (i) mixing equal volumes of oil/surfactant composition and aqueous phase; and (ii) diluting the mixture of oil/surfactant composition and aqueous phase with a further volume of aqueous phase to form a diluted composition. In a particular embodiment, the steps (i) and (ii) are each achieved by adding the oil/surfactant into the aqueous phase.

The mixing can be carried out without requiring any shear pressure, without using rotor/stator mixing, at normal pressures, and without circulating components through a pump. The method can be performed in the absence of mechanical agitation.

The mixture of the composition and the aqueous phase can be gently agitated/mixed in order to form an oil-in-water emulsion. The gentle mixing is provided by means other than homogenization, microfiltration, microfluidisation, sonication (or other high shear or high energy processes,) or a phase inversion temperature process in which the temperature of the emulsion is raised until the emulsion inverts. Suitably, the gentle agitation may comprise inversion of the mixture by hand, or it may comprise stirring, or it may comprise mixing by passing through a syringe, or it may comprise any similar process. Overall, mixing is achieved by applying controlled minimal dispersion force.

The step of combining the oil/surfactant composition and aqueous phase of a process of the invention can take place below 55° C. e.g. anywhere in the range of 5-50°, for example between 10-20° C., between 20-30° C., between 30-50° C., or between 40-50° C. The process can usefully take place at room temperature i.e. about 20-25° C. The composition and/or the aqueous phase are preferably equilibrated to the desired temperature before being mixed. For instance, the two components could be equilibrated to 40° C. and then be mixed. After mixing, the mixture can be maintained at a temperature below 55° C. while the emulsion forms. In a particular embodiment, the oil/surfactant composition and/or aqueous phase are heated before mixing and held at the desired temperature (below 55° C.) until the mixing of the two components is complete and thereafter the temperature is reduced.

The oil/surfactant composition is ideally mixed with a volume excess of the aqueous phase, to ensure that an oil-in-water emulsion is formed (rather than a water-in-oil emulsion). In a particular embodiment, the aqueous phase is substantially free from surfactant(s) and/or oil(s). The process preferably uses the aqueous phase at a volume excess of at least 5-fold e.g. between 5-fold to 50-fold greater volume. In a particular embodiment, the ratio of the aqueous phase to the oil/surfactant composition is from 10:1 to 50:1 (by volume). In another embodiment, the ratio of the aqueous phase to the oil/surfactant composition is from 20:1 to 40:1 (by volume).

The processes of the invention can be used at a lab or benchtop scale or at industrial scale. Thus the composition and/or aqueous phase may have a volume in the range of 10-100 mL, in the range of 100-1000 mL, in the range of 1-10 L, or even in the range of 10-100 L.

The methods of the invention may further comprise the step of subjecting the oil-in-water emulsion to filter sterilisation. The filter sterilisation can take place at any suitable stage e.g. when placing the emulsion into containers (the fill stage), or prior to drying (which can be performed aseptically, to maintain a sterile emulsion during and after drying).

In general, a method of the invention can involve steps of: preparing an emulsion, without using microfluidisation; filter sterilizing it; and packaging the sterilised emulsion, optionally after combining it with an immunogen. The emulsion has adjuvant activity and the packaged material is suitable for injection, for eliciting an immune response.

### The Oil-in-Water Emulsion

In a second aspect, the present invention provides an oil-in-water emulsion comprising:
a pharmaceutically acceptable oil/surfactant composition for use in the preparation of an oil-in-water emulsion having an average oil particle diameter of less than 250 nm, said composition comprising
at least one biocompatible, metabolizable oil; and
a biocompatible, metabolizable surfactant component comprising at least one surfactant;
   wherein the surfactant component has an HLB value of from 2.5 to 9.0, and wherein the oil/surfactant composition is substantially free of aqueous components; in combination with
an aqueous phase;
   wherein said emulsion has an average oil particle diameter of less than 250 nm.

Where an emulsion is described as having an average oil particle diameter of less than 250 nm, this average is ideally within the range of 100-220 nm, for example between 120-200 nm or 150-200 nm.

In a fifth aspect, the present invention provides an oil-in-water emulsion comprising:
a pharmaceutically acceptable oil/surfactant composition for use in the preparation of an oil-in-water emulsion having an average oil particle diameter of no greater than 40 nm, said composition comprising
at least one biocompatible, metabolizable oil; and
a biocompatible, metabolizable surfactant component comprising at least one surfactant;
   wherein the surfactant component has an HLB value of from 9.0 to 14.0, and wherein the oil/surfactant composition is substantially free of aqueous components; in combination with
an aqueous phase;
   wherein said emulsion has an average oil particle diameter of no greater than 40 nm.

In a particular embodiment, the average oil particle diameter of the oil-in-water emulsions according to the fifth aspect of the present invention is no greater than 30 nm, more preferably no greater than 20 nm.

The average diameter of oil particles in an emulsion can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See also reference 12. In a particular embodiment, dynamic light scattering (DLS) is the method by which oil particle diameters are determined. In a particular embodiment, the method for defining the average oil particle diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to an average diameter should be taken as an intensity-weighted average, and ideally the Z-average.

In a particular embodiment, droplets within emulsions of the invention have a polydispersity index of less than 0.4. Polydispersity is a measure of the width of the size distribution of particles, and is conventionally expressed as the polydispersity index (PdI). A polydispersity index of greater than 0.7 indicates that the sample has a very broad size distribution and a reported value of 0 means that size variation is absent, although values smaller than 0.05 are rarely seen. In a particular embodiment, it is preferred for oil droplets within an emulsion of the invention to be of a relatively uniform size. Thus, in particular embodiments, oil droplets in emulsions have a PdI of less than 0.35 e.g. less than 0.3, 0.275, 0.25, 0.225, 0.2, 0.175, 0.15, 0.125, or even less than 0.1. PdI values are easily provided by the same instrumentation which measures diameter.

### Downstream Processing

The oil-in-water emulsions of the present invention can be filtered. This filtration removes any large oil droplets from the emulsion. Although small in number terms, these oil droplets can be large in volume terms and they can act as nucleation sites for aggregation, leading to emulsion degradation during storage. Moreover, this filtration step can achieve filter sterilization.

The particular filtration membrane suitable for filter sterilization depends on the fluid characteristics of the oil-in-water emulsion and the degree of filtration required. A filter's characteristics can affect its suitability for filtration of the emulsion. For example, its pore size and surface characteristics can be important, particularly when filtering a squalene-based emulsion.

The pore size of membranes used with the invention should permit passage of the desired droplets while retaining the unwanted droplets. For example, it should retain droplets that have a size of ≧1 μm while permitting passage of droplets <200 nm. A 0.2 μm or 0.22 μm filter is ideal, and can also achieve filter sterilization.

The emulsion may be prefiltered e.g. through a 0.45 μm filter. The prefiltration and filtration can be achieved in one step by the use of known double-layer filters that include a first membrane layer with larger pores and a second membrane layer with smaller pores. Double-layer filters are particularly useful with the invention. The first layer ideally has a pore size >0.3 μm, such as between 0.3-2 μm or between 0.3-1 μm, or between 0.4-0.8 μm, or between 0.5-0.7 μm. A pore size of ≦0.75 μm in the first layer is preferred. Thus the first layer may have a pore size of 0.6 μm or 0.45 μm, for example. The second layer ideally has a pore size which is less than 75% of (and ideally less than half of) the first layer's pore size, such as between 25-70% or between 25-49% of the first layer's pore size e.g. between 30-45%, such as ⅓ or 4/9, of the first layer's pore size. Thus the second layer may have a pore size <0.3 μm, such as between 0.15-0.28 μm or between 0.18-0.24 μm e.g. a 0.2 μm or 0.22 μm pore size second layer. In one example, the first membrane layer with larger pores provides a 0.45 μm filter, while the second membrane layer with smaller pores provides a 0.22 μm filter.

The filtration membrane and/or the prefiltration membrane may be asymmetric. An asymmetric membrane is one in which the pore size varies from one side of the membrane to the other e.g. in which the pore size is larger at the entrance face than at the exit face. One side of the asymmetric membrane may be referred to as the “coarse pored surface”, while the other side of the asymmetric membrane may be referred to as the “fine pored surface”. In a double-layer filter, one or (ideally) both layers may be asymmetric.

The filtration membrane may be porous or homogeneous. A homogeneous membrane is usually a dense film ranging from 10 to 200 μm. A porous membrane has a porous structure. In one embodiment, the filtration membrane is porous. In a double-layer filter, both layers may be porous, both layers may be homogenous, or there may be one porous and one homogenous layer. A preferred double-layer filter is one in which both layers are porous.

In one embodiment, the oil-in-water emulsions of the invention are prefiltered through an asymmetric, hydrophilic porous membrane and then filtered through another asymmetric hydrophilic porous membrane having smaller pores than the prefiltration membrane. This can use a double-layer filter.

The filter membrane(s) may be autoclaved prior to use to ensure that it is sterile.

Filtration membranes are typically made of polymeric support materials such as PTFE (poly-tetra-fluoro-ethylene), PES (polyethersulfone), PVP (polyvinyl pyrrolidone), PVDF (polyvinylidene fluoride), nylons (polyamides), PP (polypropylene), celluloses (including cellulose esters), PEEK (polyetheretherketone), nitrocellulose, etc. These have varying characteristics, with some supports being intrinsically hydrophobic (e.g. PTFE) and others being intrinsically hydrophilic (e.g. cellulose acetates). However, these intrinsic characteristics can be modified by treating the membrane surface. For instance, it is known to prepare hydrophilized or hydrophobized membranes by treating them with other materials (such as other polymers, graphite, silicone, etc.) to coat the membrane surface e.g. see section 2.1 of reference 13. In a double-layer filter the two membranes can be made of different materials or (ideally) of the same material.

Details of suitable filtration techniques are available e.g. in reference 14.

During filtration, the emulsion may be maintained at a temperature of 40° C. or less, e.g. 30° C. or less, to facilitate successful sterile filtration. Some emulsions may not pass through a sterile filter when they are at a temperature of greater than 40° C.

It is advantageous to carry out the filtration step within 24 hours, e.g. within 18 hours, within 12 hours, within 6 hours, within 2 hours, within 30 minutes, of producing the emulsion because after this time it may not be possible to pass the second emulsion through the sterile filter without clogging the filter, as discussed in reference 15.

Methods of the invention may be used at large scale. Thus a method may involve filtering a volume greater than 1 liter e.g. ≧5 liters, ≧10 liters, ≧20 liters, ≧50 liters, ≧100 liters, ≧250 liters, etc.

In some embodiments, an emulsion which is prepared according to the invention can be subjected to microfluidisation. Thus, for instance, the invention can be used prior to microfluidisation to reduce the degree of microfluidising which is required for giving a desired result. Thus, if desired, microfluidisation can be used but the overall shear forces imparted on the emulsion can be reduced.

### Antigens

Although it is possible to administer oil-in-water emulsion adjuvants on their own to patients (e.g. to provide an adjuvant effect for an antigen that has been separately administered to the patient), it is more usual to admix the adjuvant with an antigen prior to administration, to form an immunogenic composition e.g. a vaccine. Mixing of emulsion and antigen may take place extemporaneously, at the time of use, or can take place during vaccine manufacture, prior to filling. The emulsions of the invention can be used in either situation.

Various antigens can be used with oil-in-water emulsions, including but not limited to: viral antigens, such as viral surface proteins; bacterial antigens, such as protein and/or saccharide antigens; fungal antigens; parasite antigens; and tumor antigens. The invention is particularly useful for vaccines against influenza virus, HIV, hookworm, hepatitis B virus, herpes simplex virus (and other herpesviridae), rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracia, Epstein Barr virus, human papillomavirus, malaria, etc. For example:

Influenza virus antigens. These may take the form of a live virus or an inactivated virus. Where an inactivated virus is used, the vaccine may comprise whole virion, split virion, or purified surface antigens (including hemagglutinin and, usually, also including neuraminidase). Influenza antigens can also be presented in the form of virosomes. The antigens may have any hemagglutinin subtype, selected from H1, H2, H3, H4, H5, H6, H7, H8, H9, H10, H11, H12, H13, H14, H15 and/or H16. Vaccine may include antigen(s) from one or more (e.g. 1, 2, 3, 4 or more) influenza virus strains, including influenza A virus and/or influenza B virus, e.g. a monovalent A/H5N1 or A/H1N1 vaccine, or a trivalent A/H1N1+A/H3N2+B vaccine. The vaccines can be for seasonal or pandemic use. The influenza virus may be a reassortant strain, and may have been obtained by reverse genetics techniques [e.g. 16-20]. Thus the virus may include one or more RNA segments from a A/PR/8/34 virus (typically 6 segments from A/PR/8/34, with the HA and N segments being from a vaccine strain, i.e. a 6:2 reassortant). The viruses used as the source of the antigens can be grown either on eggs (e.g. embryonated hen eggs) or on cell culture. Where cell culture is used, the cell substrate will typically be a mammalian cell line, such as MDCK; CHO; 293T; BHK; Vero; MRC-5; PER.C6; WI-38; etc. Preferred mammalian cell lines for growing influenza viruses include: MDCK cells [21-24], derived from Madin Darby canine kidney; Vero cells [25-27], derived from African green monkey kidney; or PER.C6 cells [28], derived from human embryonic retinoblasts. Where virus has been grown on a mammalian cell line then the composition will advantageously be free from egg proteins (e.g. ovalbumin and ovomucoid) and from chicken DNA, thereby reducing allergenicity. Unit doses of vaccine are typically standardized by reference to hemagglutinin (HA) content, typically measured by SRID. Existing vaccines typically contain about 15 μg of HA per strain, although lower doses can be used, particularly when using an adjuvant. Fractional doses such as ½ (i.e. 7.5 μg HA per strain), ¼ and ⅛ have been used [29,30], as have higher doses (e.g. 3× or 9× doses [31,32]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 15, about 10, about 7.5, about 5, about 3.8, about 3.75, about 1.9, about 1.5, etc. per strain.

Human immunodeficiency virus, including HIV-1 and HIV-2. The antigen will typically be an envelope antigen.

Hepatitis B virus surface antigens. This antigen is preferably obtained by recombinant DNA methods e.g. after expression in a Saccharomyces cerevisiae yeast. Unlike native viral HBsAg, the recombinant yeast-expressed antigen is non-glycosylated. It can be in the form of substantially-spherical particles (average diameter of about 20 nm), including a lipid matrix comprising phospholipids. Unlike native HBsAg particles, the yeast-expressed particles may include phosphatidylinositol. The HBsAg may be from any of subtypes ayw1, ayw2, ayw3, ayw4, ayr, adw2, adw4, adrq− and adrq+.

Hookworm, particularly as seen in canines (Ancylostoma caninum). This antigen may be recombinant Ac-MTP-1 (astacin-like metalloprotease) and/or an aspartic hemoglobinase (Ac-APR-1), which may be expressed in a baculovirus/insect cell system as a secreted protein [33,34].

Herpes simplex virus antigens (HSV). A preferred HSV antigen for use with the invention is membrane glycoprotein gD. It is preferred to use gD from a HSV-2 strain (‘gD2’ antigen). The composition can use a form of gD in which the C-terminal membrane anchor region has been deleted [35] e.g. a truncated gD comprising amino acids 1-306 of the natural protein with the addition of aparagine and glutamine at the C-terminus. This form of the protein includes the signal peptide which is cleaved to yield a mature 283 amino acid protein. Deletion of the anchor allows the protein to be prepared in soluble form. The invention can also be used with other herpesviridae, such as varicella-zoster virus (VZV), Epstein-Barr virus (EBV), or human cytomegalovirus (hCMV). An anti-hCMV composition can include a glycoprotein B (gB) antigen in some embodiments, or can include one or more of the gH, gL and gO antigens.

Human papillomavirus antigens (HPV). Preferred HPV antigens for use with the invention are L1 capsid proteins, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L1 gene(s). More preferably, the composition includes L1 VLPs from both HPV-16 and HPV-18 strains. This bivalent combination has been shown to be highly effective [36]. In addition to HPV-16 and HPV-18 strains, it is also possible to include L1 VLPs from HPV-6 and HPV-11 strains. The use of oncogenic HPV strains is also possible. A vaccine may include between 20-60 μg/ml (e.g. about 40 μg/ml) of L1 per HPV strain.

Anthrax antigens. Anthrax is caused by Bacillus anthracia. Suitable B. anthracis antigens include A-components (lethal factor (LF) and edema factor (EF)), both of which can share a common B-component known as protective antigen (PA). The antigens may optionally be detoxified. Further details can be found in references [37 to 39].

Malaria antigens. A composition for protecting against malaria can include a portion of the P. falciparum circumsporozoite protein from the organism's pre-erythrocytic stage. The C-terminal portion of this antigen can be expressed as a fusion protein with HBsAg, and this fusion protein can be co-expressed with HBsAg in yeast such that the two proteins assemble to form a particle.

Rabies. Compositions for protecting against rabies will generally include an inactivated rabies virus virion, as seen in products such as RABIPUR, RABIVAC, and VERORAB.

S. aureus antigens. A variety of S. aureus antigens are known. Suitable antigens include capsular saccharides (e.g. from a type 5 and/or type 8 strain) and proteins (e.g. IsdB, Hla, etc.). Capsular saccharide antigens are ideally conjugated to a carrier protein.

S. pneumoniae antigens. A variety of S. pneumoniae antigens are known. Suitable antigens include capsular saccharides (e.g. from one or more of serotypes 1, 4, 5, 6B, 7F, 9V, 14, 18C, 19F, and/or 23F) and proteins (e.g. pneumolysin, detoxified pneumolysin, polyhistidine triad protein D (PhtD), etc.). Capsular saccharide antigens are ideally conjugated to a carrier protein.

Meningococcal antigens. Neisseria meningitidis is a cause of bacterial meningitis. Suitable meningococcal antigens include conjugated capsular saccharides (particularly for serogroups A, C, W135, X and/or Y), recombinant proteins (e.g. factor H binding protein) and/or outer membrane vesicles.

Cancer antigens. A variety of tumour-specific antigens are known. The invention may be used with antigens that elicit an immunotherapeutic response against lung cancer, melanoma, breast cancer, prostate cancer, etc.

A solution of the antigen will normally be mixed with the emulsion e.g. at a 1:1 volume ratio. This mixing can either be performed by a vaccine manufacturer, prior to filling, or can be performed at the point of use, by a healthcare worker. As noted below, however, an alternative formulation includes both antigen and emulsion in dried form in a single container for reconstitution.

### Uses of the Oil-in-Water Emulsions of the Invention

According to one aspect of the present invention the oil-in-water emulsions of the present invention are for use as an adjuvant, preferably a vaccine adjuvant. Suitably, said adjuvant is administered as part of a vaccine.

In a further aspect the present invention provides an immunogenic composition (e.g. a vaccine) comprising an oil-in-water emulsion according to the present invention, and an antigen component. The invention further provides a process for preparing such immunogenic compositions, said process comprising mixing an oil-in-water emulsion according to the present invention, with an antigen component.

The present invention also provides a kit comprising:
an oil/surfactant composition according to the present invention;
an aqueous phase; and optionally
instructions for combining the oil/surfactant composition and aqueous phase.

According to embodiments of the invention the oil/surfactant composition and/or the aqueous phase may comprise an antigen component.

In a further aspect the present invention provides a process for preparing a kit comprising the steps of:
providing an oil/surfactant composition according to the present invention; and
packaging the composition into a kit as a kit component together with an aqueous phase; and optionally
packaging an antigen component into the kit as a kit component together with the oil/surfactant composition and the aqueous phase.

The present invention also provides a kit comprising:
an oil-in-water emulsion according to the present invention; and
an antigen component

The present invention further provides a process for preparing a kit comprising the steps of:
providing an oil-in-water emulsion according to the present invention; and
packaging the emulsion into a kit as a kit component together with a separate antigen component.

Although it is possible to administer oil in water emulsion adjuvants on their own to patients (e.g. to provide an adjuvant effect for an antigen that has been separately administered to the patient), it is more usual to admix the adjuvant with an antigen prior to administration, to form an immunogenic composition e.g. a vaccine. Mixing of emulsion and antigen may take place extemporaneously, at the time of use, or can take place during vaccine manufacture, prior to filling.

Overall, therefore, the invention can be used when preparing mixed vaccines or when preparing kits including antigen and adjuvant ready for mixing. Where mixing takes place during manufacture then the volumes of bulk antigen and emulsion that are mixed will typically be greater than 1 liter e.g. ≧5 liters, ≧10 liters, ≧20 liters, ≧50 liters, ≧100 liters, ≧250 liters, etc. Where mixing takes place at the point of use then the volumes that are mixed will typically be smaller than 1 milliliter e.g. ≦0.6 ml, ≦0.5 ml, ≦0.4 ml, ≦0.3 ml, ≦0.2 ml, etc. In both cases it is usual for substantially equal volumes of emulsion and antigen solution to be mixed i.e. substantially 1:1 (e.g. between 1.1:1 and 1:1.1, preferably between 1.05:1 and 1:1.05, and more preferably between 1.025:1 and 1:1.025). In some embodiments, however, an excess of emulsion or an excess of antigen may be used [40]. Where an excess volume of one component is used, the excess will generally be at least 1.5:1 e.g. ≧2:1, ≧2.5:1, ≧3:1, ≧4:1, ≧5:1, etc.

Where antigen and adjuvant are presented as separate components within a kit, they are physically separate from each other within the kit, and this separation can be achieved in various ways. For instance, the components may be in separate containers, such as vials. The contents of two vials can then be mixed when needed e.g. by removing the contents of one vial and adding them to the other vial, or by separately removing the contents of both vials and mixing them in a third container.

In another arrangement, one of the kit components is in a syringe and the other is in a container such as a vial. The syringe can be used (e.g. with a needle) to insert its contents into the vial for mixing, and the mixture can then be withdrawn into the syringe. The mixed contents of the syringe can then be administered to a patient, typically through a new sterile needle. Packing one component in a syringe eliminates the need for using a separate syringe for patient administration.

In another preferred arrangement, the two kit components are held together but separately in the same syringe e.g. a dual-chamber syringe, such as those disclosed in references 41-48 etc. When the syringe is actuated (e.g. during administration to a patient) then the contents of the two chambers are mixed. This arrangement avoids the need for a separate mixing step at time of use.

The contents of the various kit components will generally all be in liquid form.

Vaccines are typically administered by injection, particularly intramuscular injection. Compositions of the invention are generally presented at the time of use as aqueous solutions or suspensions. In some embodiments of the invention the compositions are in aqueous form from the packaging stage to the administration stage. In other embodiments, however, one or more components of the compositions may be packaged in dried (e.g. lyophilised) form, and an adjuvant for actual administration may be reconstituted when necessary. The emulsion may thus be distributed as a lyophilized cake. Drying can conveniently be achieved by lyophilisation, but other techniques can also be used e.g. spray drying. Drying by lyophilisation is preferred.

Accordingly in one aspect the present invention provides a dried material (e.g. a lyophilisate) which, when reconstituted with an aqueous phase provides an oil-in-water emulsion according to the present invention. The dried material is preferably a lyophilisate. As used herein, “dry material” or “dried material” refers to material which is substantially free of water or material which is substantially free of an aqueous phase. Ideally an emulsion can be reconstituted to its original composition merely by adding water (e.g. it does not lose any components except water during drying). The invention also provides a process for preparing said dried material wherein said process comprises preparing an oil-in-water emulsion according to the invention and subjecting the emulsion to a drying process. Suitably the emulsion is combined with one or more lyophilisation stabilizers prior to lyophilisation. The emulsion may also be combined with at least one antigen component prior to drying, optionally in addition to one or more lyophilisation stabilizers.

In some arrangements, a component (typically the emulsion component) is in dry form (e.g. in a lyophilized form), with the remaining components (typically the antigen and/or an aqueous phase) being in liquid form. The two or more components can be mixed in order to reactivate the dry component and give a liquid composition for administration to a patient. A dried component will typically be located within a vial rather than a syringe.

A lyophilised component (e.g. the emulsion) may include lyophilisation stabilizers. These stabilizers include substances such as sugar alcohols (e.g. mannitol, etc.) or simple saccharides such as disaccharides and trisaccharides. Lyophilisation stabilizers are preferably small saccharides such as disaccharides. They preferably include saccharide monomers selected from glucose, fructose and galactose, and glucose-containing disaccharides and fructose-containing disaccharides are particularly preferred. Examples of preferred disaccharides include sucrose (containing glucose and fructose), trehalose (containing two glucose monosaccharides) and maltulose (containing glucose and fructose), more preferably sucrose, such as lactose, sucrose or mannitol, as well as mixtures thereof e.g. lactose/sucrose mixtures, sucrose/mannitol mixtures, etc.

In a particular embodiment, one possible arrangement comprises a dried emulsion component in a vial and an antigen component and/or aqueous phase in a pre-filled syringe.

The present invention also provides an arrangement comprising a dried emulsion of the present invention and a separate liquid antigen component.

Also provided by the present invention is a dried cake formed from the emulsion of the invention. The cake may be provided in combination with a separate aqueous phase. The arrangement may further comprise an antigen component which may be in liquid or dried form.

The present invention also provides a dried mixture wherein the mixture comprises the emulsion of the present invention in combination with an antigen component. In a particular embodiment, the mixture is a lyophilized mixture.

An advantage of the oil-in-water emulsions of the invention and the methods for making the same according to the invention is that when the oil-in-water emulsion is reconstituted with an aqueous phase following drying of the emulsion, the resultant oil-in-water emulsion retains its original properties from prior to drying (e.g. its average oil particle diameter).

The invention also provides a kit for preparing an oil-in-water emulsion of the invention, wherein the kit comprises an oil-in-water emulsion of the invention in dried form and an aqueous phase in liquid form. The kit may comprises two vials (one containing the dried emulsion and one containing the aqueous phase) or it may comprise one ready filled syringe and one vial e.g. with the contents of the syringe (the aqueous phase) being used to reconstitute the contents of the vial (the dried emulsion) prior to administration to a subject. In embodiments of the invention the oil-in-water emulsion in dried form is combined with an antigen component that is also in dried form.

If vaccines contain components in addition to emulsion and antigen then these further components may be included in one of the two kit components according to embodiments of the invention, or may be part of a third kit component.

Suitable containers for mixed vaccines of the invention, or for individual kit components, include vials and disposable syringes. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. In one embodiment, a vial has a butyl rubber stopper. The vial may include a single dose of vaccine/component, or it may include more than one dose (a ‘multidose’ vial) e.g. 10 doses. In one embodiment, a vial includes 10×0.25 ml doses of emulsion. Preferred vials are made of colourless glass.

A vial can have a cap (e.g. a Luer lock) adapted such that a pre-filled syringe can be inserted into the cap, the contents of the syringe can be expelled into the vial (e.g. to reconstitute dried material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Where a composition/component is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of adjuvant or vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield.

The emulsion may be diluted with a buffer prior to packaging into a vial or a syringe. Typical buffers include: a phosphate buffer; a Tris buffer; a borate buffer; a succinate buffer; a histidine buffer; or a citrate buffer. Dilution can reduce the concentration of the adjuvant's components while retaining their relative proportions e.g. to provide a “half-strength” adjuvant.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

Compositions made using the methods of the invention are pharmaceutically acceptable. They may include components in addition to the emulsion and the optional antigen.

The composition may include a preservative such as thiomersal or 2-phenoxyethanol. It is preferred, however, that the adjuvant or vaccine should be substantially free from (i.e. less than 5 μg/ml) mercurial material e.g. thiomersal-free [49,50]. Vaccines and components containing no mercury are more preferred.

The pH of a composition will generally be between 5.0 and 8.1, and more typically between 6.0 and 8.0 e.g. between 6.5 and 7.5. A process of the invention may therefore include a step of adjusting the pH of the adjuvant or vaccine prior to packaging.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunization, or may include material for multiple immunizations (i.e. a ‘multidose’ kit). The inclusion of a preservative is preferred in multidose arrangements.

The compositions can be administered in various ways. The most preferred immunization route is by intramuscular injection (e.g. into the arm or leg), but other available routes include subcutaneous injection, intranasal [51-53], oral [54], intradermal [55,56], transcutaneous, transdermal [57], etc.

Adjuvants or vaccines prepared according to the invention may be used to treat both children and adults. The patient may be less than 1 year old, 1-5 years old, 5-15 years old, 15-55 years old, or at least 55 years old. The patient may be elderly (e.g. ≧50 years old, preferably ≧65 years), the young (e.g. ≦5 years old), hospitalized patients, healthcare workers, armed service and military personnel, pregnant women, the chronically ill, immunodeficient patients, and people travelling abroad. The vaccines are not suitable solely for these groups, however, and may be used more generally in a population.

Adjuvants or vaccines of the invention may be administered to patients at substantially the same time as (e.g. during the same medical consultation or visit to a healthcare professional) other vaccines.

### DEFINITIONS

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

### MODES FOR CARRYING OUT THE INVENTION

The examples set out below are for illustrative purposes and are not intended to limit the scope of the invention.

### 1/ Formation of Oil-in-Water Emulsion and Measurement of the Average Particle Size

Oil/surfactant compositions comprising squalene, Span 85 and Tween 80 were prepared with the percentage compositions set out in tables 1 & 2. These compositions were mixed at 37-40° C. overnight. The following day the oil compositions were combined with aqueous material by adding the oil/surfactant composition to the aqueous material in a 1:10 (oil/surfactant to water) (volume/volume) ratio at room temperature; it was observed that oil-in-water emulsions formed spontaneously. The average oil particle size of various emulsions was measured using a Zetasizer Nano ZS (Malvern Instruments) according to the manufacturer's instructions (to give a Z-average and a PdI), or a Wyatt DynaPro high-throughput particle sizer (by intensity). The results of this study are set out in Tables 1 and 2.

TABLE 1

| | | | | Oil | | |
|---|---|---|---|---|---|---|
| | | | | particle | | High- |
| | | | Tween | Z-average | | thruput |
| Emulsion | Squalene | Span 85 | 80 | diameter | | diameter |
| ID | (% vol.) | (% vol.) | (% vol.) | (nm) | PdI | (nm) |
| SEA01 | 20 | 70 | 10 | | | 150.1 |
| SEA04 | 30 | 50 | 20 | | | 128.7 |
| SEA05 | 40 | 40 | 20 | 43.15 | 0.1 | |
| SEA06 | 30 | 40 | 30 | 94.87 | 0.069 | |
| SEA08 | 40 | 50 | 10 | | | 202.4 |
| SEA10 | 50 | 30 | 20 | 172 | 0.055 | |
| SEA11 | 40 | 30 | 30 | 115.4 | 0.138 | |
| SEA15 | 20 | 60 | 20 | | | 221.0 |
| SEA25 | 10 | 50 | 40 | | | 191.3 |
| SEA26 | 10 | 60 | 30 | | | 206.5 |
| SEA27 | 10 | 70 | 20 | | | 196.7 |
| SEA28 | 10 | 80 | 10 | | | 217.1 |

TABLE 2

| | | | | Oil | | |
|---|---|---|---|---|---|---|
| | | | | particle | | High- |
| | | | Tween | Z-average | | throughput |
| Emulsion | Squalene | Span 85 | 80 | diameter | | diameter |
| ID | (% vol.) | (% vol.) | (% vol.) | (nm) | PdI | (nm) |
| SEA12 | 30 | 30 | 40 | 23.19 | 0.144 | 25.2 |
| SEA19 | 40 | 20 | 40 | 19.48 | 0.146 | 20.5 |
| SEA20 | 30 | 20 | 50 | 17.48 | 0.142 | 18.2 |
| SEA29 | 10 | 10 | 80 | 11.32 | 0.104 | 11.3 |

Oil/surfactant compositions comprising squalene, Span 85 and Tween 80 were prepared with the percentage compositions set out in Table 3 below. These compositions were combined with aqueous material by adding the oil/surfactant composition to the aqueous material in a 1:10 (oil/surfactant to water) (volume/volume) ratio at room temperature and it was observed that oil-in-water emulsions were not formed spontaneously or an emulsion was formed but having an average oil particle size of 250 nm or greater. The average oil particle size of various emulsions was measured using a Wyatt DynaPro high-throughput particle sizer (by intensity) according to the manufacturer's instructions.

TABLE 3

| Emulsion | Squalene | Span 85 | Tween 80 | Average oil particle |
|---|---|---|---|---|
| ID | (% vol.) | (% vol.) | (% vol.) | diameter (nm) |
| SEA07 | 30 | 60 | 10 | — |
| SEA09 | 50 | 40 | 10 | 251.4 |
| SEA13 | 20 | 40 | 40 | — |
| SEA14 | 20 | 50 | 30 | — |
| SEA16 | 70 | 20 | 10 | — |
| SEA17 | 60 | 20 | 20 | — |
| SEA18 | 50 | 20 | 30 | — |
| SEA21 | 20 | 20 | 60 | — |
| SEA22 | 10 | 20 | 70 | — |
| SEA23 | 10 | 30 | 60 | — |
| SEA24 | 10 | 40 | 50 | 289.4 |
| SEA30 | 20 | 10 | 70 | 350.3 |
| SEA31 | 30 | 10 | 60 | 423.6 |
| SEA32 | 40 | 10 | 50 | 340.3 |
| SEA33 | 50 | 10 | 40 | — |
| SEA34 | 60 | 10 | 30 | — |
| SEA35 | 70 | 10 | 20 | — |
| SEA36 | 80 | 10 | 10 | 316.2 |

As shown in Table 1, oil-in-water emulsions according to the present invention may have an average oil particle size of less than 200 nm. The oil-in-water emulsions according to certain embodiments of the invention may have an average oil particle size of no greater than 40 nm as shown in Table 2.

### 2/ Formation of Oil-in-Water Emulsion and Measurement of the Average Particle Size.

400 μl of squalene, 400 μl of Span 85 (sorbitan trioleate), and 200 μl of Tween 80 (polysorbate 80; polyoxyethylene sorbitan monooleate) were mixed together to form a 1 mL composition according to the present invention. Following formation of the oil and surfactant containing composition, said composition was added to 19 mL of an aqueous phase comprising water to form a 20 mL mixture. The resulting mixture was then inverted gently by hand, resulting in the formation of an oil-in-water emulsion according to the present invention. The average oil droplet particle size in the oil-in-water emulsion was measured as 43.15 nm.

This experiment was repeated with various different combinations of squalene, Span 85 and Tween 80 according to the present invention. The results of these experiments are set out in Table 4 below. For reference, the corresponding data for the known adjuvant MF59 is also provided in Table 4. The average oil particle diameters of each emulsion were measured using a Zetasizer Nano ZS (Malvern Instruments, Worcestershire, UK) according to the manufacturer's instructions.

TABLE 4

| | | | | Squalene | Span 85 | Tween 80 | Particle |
|---|---|---|---|---|---|---|---|
| Emulsion | Squalene | Span | Tween | compared | compared | compared | diameter |
| ID | (μl) | 85 (μl) | 80 (μl) | to MF59 | to MF59 | to MF59 | (nm) |
| SEA05 | 400 | 400 | 200 | 40% | 400% | 200% | 43.15 |
| SEA10 | 500 | 300 | 200 | 50% | 300% | 200% | 172 |
| SEA11 | 400 | 300 | 300 | 40% | 300% | 300% | 115.4 |
| SEA19 | 400 | 200 | 400 | 40% | 200% | 400% | 19.48 |
| MF59 | 1000 | 100 | 100 | 100% | 100% | 100% | ~160 |

As demonstrated in Table 4, each of the emulsions tested had an average oil particle size of less than 175 nm. In particular, emulsion SEA19 was found to have a squalene particle size of less than 20 nm.

Accordingly, the present invention allows for the manufacture of highly dispersed oil-in-water emulsions having an average oil particle diameter of less than 250 nm. The present invention also allows for the manufacture of highly dispersed oil-in-water emulsions with very small oil (squalene) particles having an average oil particle diameter of no greater than 40 nm. Furthermore, the small particle sizes are achieved without the need for expensive processes such as microfluidization.

Table 4 also shows the relative percentages of each component in the emulsion compared to the corresponding amount of each component in the known vaccine adjuvant MF59. For example, the composition SEA19 of the present invention contains only two fifths of the amount of squalene present in MF59, twice as much Span 85, and four times as much Tween 80 as MF59, and has an average particle diameter which is over eight times smaller than that in MF59. Generally, each of the emulsions tested in example 2 contained less than or equal to half the amount of squalene present in the vaccine adjuvant MF59. Furthermore, the emulsions of the present invention tested in example 2 contain anywhere between two and four times the amount of each surfactant found in MF59.

3/ In Vivo Testing of Oil-in-Water Emulsions for their Potential Use as Adjuvants

In this experiment, the oil-in-water emulsion SEA19 of example 2 was tested in vivo for its ability to enhance an immune response in a subject. Comparative experiments were also carried out in which no adjuvant or the known adjuvant MF59 was administered in place of the emulsion SEA19. Furthermore, varying dose concentrations of the emulsion SEA19 were tested.

Each experiment listed in Table 5 was carried out on two groups of mice, each group consisting of four mice. Each group was administered 1 or 10 μg of the influenza (Brisbane strain) antigens in combination with the adjuvant MF59, SEA19 or no adjuvant in accordance with the experiments set out in Table 5 below.

TABLE 5

| Adjuvant | Adjuvant | Antigen | |
|---|---|---|---|
| Name | Dose | Dose | Average ELISA |
| (None) | — | 10 μg | 28686 |
| (None) | — | 1 μg | 14599 |
| MF59 | 1x | 10 μg | 267761 |
| MF59 | 1x | 1 μg | 170368 |
| SEA19 | 1x | 10 μg | 99058 |
| SEA19 | 1x | 1 μg | 104406 |
| SEA19 | 0.1x | 10 μg | 42053 |
| SEA19 | 0.1x | 1 μg | 25392 |
| SEA19 | 0.01x | 10 μg | 48296 |
| SEA19 | 0.01x | 1 μg | 21174 |

As demonstrated in Table 5, the SEA19 emulsion of the present invention was effective in inducing immune response against the influenza antigen. Accordingly, the emulsions of the present invention are suitable for use as adjuvants, for example in vaccines.

### 4/ Alternative Oils and Surfactants

The composition of SEA20 as discussed above was defined more precisely based on a volume ratio of 600:200:1400 (squalene:sorbitan trioleate:polysorbate 80), thus leading to 27.3% by volume squalene, 9.1% sorbitan trioleate, and 63.6% polysorbate 80. When combined with a 19-fold excess of aqueous material (1.1 mL of mixture with 19 mL of 100 mM citrate buffer, pH 6.5) this SEA20 mixture gives an emulsion with droplets ˜20 nm in diameter, with a PdI of ˜0.1.

Whereas the three components in SEA20 have a volume excess of surfactant, the same components can be combined in different ratios with a volume excess of squalene. In contrast to SEA20, with 70% by volume squalene this mixture provides an emulsion with droplets ˜160 nm in diameter, also with a PdI of ˜0.1.

The oil and surfactants in these two emulsions were varied. Three alternative oils were used (squalane, soybean oil, sunflower seed oil) and three alternative high-HLB surfactants (polysorbate 20, HLB 16.7; SDS, HLB 40; or polyoxyethylene (10) lauryl ether, HLB 17) at the same volumes (except for SDS, which was used at the same weight). These alternative emulsions had the following characteristics:

| | | | | | |
|---|---|---|---|---|---|
| | Span 85 | | Diameter | | Stability |
| Oil (μL) | (μL) | High HLB (μL) | (nm) | PdI | comments |

SEA20 and variations

| | | | | | | | |
|---|---|---|---|---|---|---|---|
| Squalene | 600 | 200 | Polysorbate 80 | 1400 | ~20 | ~0.09 | Clear |
| Squalane | 600 | 200 | Polysorbate 80 | 1400 | 704.7 | 0.759 | Turbid, creaming |
| Soybean | 600 | 200 | Polysorbate 80 | 1400 | 126.3 | 0.285 | Turbid, creaming |
| Sunflower | 600 | 200 | Polysorbate 80 | 1400 | 180.8 | 0.385 | Turbid |
| Squalene | 600 | — | Polysorbate 80 | 1600 | 15.14 | 0.068 | Clear |
| Squalene | 600 | — | Polysorbate 80 | 1400 | 79.44 | 0.418 | Turbid |
| Squalene | 600 | — | Polysorbate 20 | 1600 | 941.1 | 1 | Milky |
| Squalene | 600 | 200 | Polysorbate 20 | 1400 | 477.4 | 0.93 | Milky |
| Squalene | 600 | — | Polysorbate 20 | 1400 | 789.8 | 0.622 | Turbid |
| Squalene | 600 | 200 | SDS | 756 | 432.3 | 0.477 | Turbid |
| Squalene | 300 | — | SDS | 756 | 2598 | 0.519 | Turbid |
| Squalene | 600 | 200 | POE 10 LE | 1400 | — | — | Oil phase = paste |

Alternative mixture, and variations

| | | | | | | | |
|---|---|---|---|---|---|---|---|
| Squalane | 1400 | 300 | Polysorbate 80 | 300 | 387.3 | 0.491 | Milky |
| Soybean | 1400 | 300 | Polysorbate 80 | 300 | 1046 | 0.659 | Milky |
| Sunflower | 1400 | 300 | Polysorbate 80 | 300 | 650.8 | 0.709 | Milky |
| Squalene | 1400 | — | Polysorbate 80 | 600 | 2530 | 1 | Creaming |
| Squalene | 1400 | — | Polysorbate 80 | 300 | 1757 | 0.995 | Creaming |
| Squalene | 1400 | — | Polysorbate 20 | 600 | 1478 | 0.777 | Creaming |
| Squalene | 1400 | 300 | Polysorbate 20 | 300 | 487 | 0.561 | Turbid, creaming |
| Squalene | 1400 | — | Polysorbate 20 | 300 | 1517 | 0.991 | Creaming |
| Squalene | 1400 | 300 | SDS | 162 | 1081 | 0.832 | Turbid |
| Squalene | 700 | — | SDS | 162 | 3183 | 0.792 | Turbid |
| Squalene | 1400 | 300 | POE 10 LE | 300 | — | — | Separated |

Thus none of the alternative oils was a suitable substitute for squalene, and none of the alternative surfactant components was useful either.

It will be understood that the invention has been described by way of example only and modifications may be made whilst remaining within the scope and spirit of the invention. The embodiments within the specification provide an illustration of embodiments of the invention and should not be construed to limit the scope of the invention. The skilled artisan readily recognizes that many other embodiments are encompassed by the invention.

### REFERENCES

[1] WO90/14837.
[2] Podda & Del Giudice (2003) Expert Rev Vaccines 2:197-203.
[3] Podda (2001) Vaccine 19: 2673-2680.
[4] Vaccine Design: The Subunit and Adjuvant Approach (eds. Powell & Newman) Plenum Press 1995 (ISBN 0-306-44867-X).
[5] Vaccine Adjuvants: Preparation Methods and Research Protocols (Volume 42 of Methods in Molecular Medicine series). ISBN: 1-59259-083-7. Ed. O'Hagan.
[6] New Generation Vaccines (eds. Levine et al.). 3rd edition, 2004. ISBN 0-8247-4071-8.
[7] O'Hagan (2007) Expert Rev Vaccines 6(5):699-710.
[8] Garçon et al. (2012) Expert Rev Vaccines 11:349-66.
[9] WO2006/100109.
[10] US2007/0014805.
[11] Ott et al. (2000) Chapter 12 of Vaccine Adjuvants: Preparation methods and research protocols (ed. O'Hagan), volume 42 of Methods in Molecular Medicine.
[12] Light Scattering from Polymer Solutions and Nanoparticle Dispersions (W. Schartl), 2007. ISBN: 978-3-540-71950-2.
[13] WO90/04609.
[14] WO2011/067669
[15] Lidgate et al (1992) Pharmaceutical Research 9(7):860-863.
[16] Hoffmann et al. (2002) Vaccine 20:3165-3170.
[17] Subbarao et al. (2003) Virology 305:192-200.
[18] Liu et ah (2003) Virology 314:580-590.
[19] Ozaki et al. (2004) J. Virol. 78:1851-1857.
[20] Webby et al. (2004) Lancet 363:1099-1103.
[21] WO97/37000.
[22] Brands et al. (1999) Dev Biol Stand 98:93-100.
[23] Halperin et al. (2002) Vaccine 20:1240-7.
[24] Tree et al. (2001) Vaccine 19:3444-50.
[25] Kistner et al. (1998) Vaccine 16:960-8.
[26] Kistner et al. (1999) Dev Biol Stand 98:101-110.
[27] Bruhl et al. (2000) Vaccine 19:1149-58.
[28] Pau et al. (2001) Vaccine 19:2716-21.
[29] WO01/22992.
[30] Hehme et al. (2004) Virus Res. 103(1-2):163-71.
[31] Treanor et al. (1996) J Infect Dis 173:1467-70.
[32] Keitel et al. (1996) Clin Diagn Lab Immunol 3:507-10.
[33] Williamson et al. (2006) Infection and Immunity 74: 961-7.
[34] Loukas et al. (2005) PLoS Med 2(10): e295.
[35] EP-A-0139417.
[36] Harper et al. (2004) Lancet 364(9447):1757-65.
[37] J Toxicol Clin Toxicol (2001) 39:85-100.
[38] Demicheli et al. (1998) Vaccine 16:880-884.
[39] Stepanov et al. (1996) J Biotechnol 44:155-160.
[40] WO2007/052155.
[41] WO2005/089837.
[42] U.S. Pat. No. 6,692,468.
[43] WO00/07647.
[44] WO99/17820.
[45] U.S. Pat. No. 5,971,953.
[46] U.S. Pat. No. 4,060,082.
[47] EP-A-0520618.
[48] WO98/01174.
[49] Banzhoff (2000) Immunology Letters 71:91-96.
[50] WO02/097072.
[51] Greenbaum et al. (2004) Vaccine 22:2566-77.
[52] Zurbriggen et al. (2003) Expert Rev Vaccines 2:295-304.
[53] Piascik (2003) J Am Pharm Assoc (Wash DC). 43:728-30.
[54] Mann et al. (2004) Vaccine 22:2425-9.
[55] Halperin et al. (1979) Am J Public Health 69:1247-50.
[56] Herbert et al. (1979) J Infect Dis 140:234-8.
[57] Chen et al. (2003) Vaccine 21:2830-6.

1. A pharmaceutically acceptable oil/surfactant composition for use in the preparation of an oil-in-water emulsion having an average oil particle diameter of less than 250 nm, said composition comprising
(i) squalene; and
(ii) a biocompatible, metabolizable surfactant component comprising at least one surfactant, wherein: (a) the surfactant component has an HLB value of from 2.5 to 9.0; (b) the surfactant component has an HLB value of from 3.3 to 8.4; (c) the surfactant component has an HLB value of from 10 to 18; (d) the surfactant component has an HLB value of from 13 to 17; (e) the surfactant component has an HLB value of from 14 to 16; or (f) the surfactant component has an HLB value of from 9 to 14,
wherein the oil/surfactant composition is substantially free of aqueous components.

2. The oil/surfactant composition of claim 1, wherein the surfactant component comprises or consists of polysorbate 80.

3. The oil/surfactant composition of claim 1, wherein: (a) the surfactant component has an HLB value of from 2.5 to 9.0 and the oil-in-water emulsion has an average oil particle diameter of between 40-250 nm; or (b) the surfactant component has an HLB value of from 9.0 to 14.0 and the oil-in-water emulsion has an average oil particle diameter of no greater than 40 nm.

4. The oil/surfactant composition of claim 1, wherein: (a) the composition includes a volume excess of squalene relative to the surfactant component; or (b) the composition includes a volume excess of the surfactant component relative to squalene.

5. The oil/surfactant composition of claim 1, comprising (by volume) 60-80% squalene (for instance, 65-75% squalene, or 70% squalene), with the remainder being polysorbate 80 or a combination of polysorbate 80 and sorbitan trioleate.

6. The oil/surfactant composition of claim 1, wherein (a) the composition includes squalene and polysorbate 80, and the combined % by volume of squalene and polysorbate 80 is from 70 to 90% of the total volume of the oil/surfactant composition; or (b) the composition includes squalene, sorbitan trioleate and polysorbate 80, and the combined % by volume of squalene and polysorbate 80 is from 70 to 90% of the total volume of squalene, sorbitan trioleate and polysorbate 80 in the oil/surfactant composition.

7. An oil-in-water emulsion comprising the oil/surfactant composition of claim 1 in combination with an aqueous phase, wherein said emulsion has an average oil particle diameter of less than 250 nm, preferably within the range of 20-200 nm.

8. A method of forming an oil-in-water emulsion comprising
(i) squalene; and
(ii) a biocompatible, metabolizable surfactant component comprising at least one surfactant;
wherein: (a) the surfactant component has an HLB value of from 2.5 to 9.0; (b) the surfactant component has an HLB value of from 3.3 to 8.4; (c) the surfactant component has an HLB value of from 10 to 18; (d) the surfactant component has an HLB value of from 13 to 17; (e) the surfactant component has an HLB value of from 14 to 16; or (f) the surfactant component has an HLB value of from 9 to 14, and wherein the emulsion has an average oil particle diameter of less than 250 nm;
said method comprising:
a) providing an oil/surfactant composition according to claim 1;
b) providing an aqueous phase;
c) combining the oil/surfactant composition with a volume excess of the aqueous phase to form a diluted composition; and
d) gently mixing the diluted composition to form an oil-in-water emulsion having an average oil particle diameter of less than 250 nm.

9. (canceled)

10. An immunogenic composition comprising an oil-in-water emulsion according to claim 7, and an antigen component.

11. A process for preparing an immunogenic composition comprising mixing an oil-in-water emulsion according to claim 7, with an antigen component.

12. A kit comprising:
(i) an oil/surfactant composition according to claim 1;
(ii) an aqueous phase; and optionally
(iii) instructions for combining the oil/surfactant composition and aqueous phase.

13. A dried material which, when reconstituted with an aqueous phase provides an oil-in-water emulsion according to claim 7.

14. A kit for preparing an oil-in-water emulsion according to claim 7, wherein the kit comprises:
(i) an oil-in-water emulsion according to claim 7 in dried form; and
(ii) an aqueous phase.

15. The method of claim 8, wherein the surfactant component comprises or consists of polysorbate 80.

16. A kit comprising:
(ii) an oil-in-water emulsion according to claim 7; and
(iii) an antigen component.
